(19) United States
(12) Patent Application Publication
(10) Pub. No.: US 2007/0179574 A1
(43) Pub. Date: Aug. 2, 2007

(54) Apparatus for phototherapeutic treatment of skin disorders

(76) Inventor: Donald Bruce Elliott, Barrie (CA)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 11/342,908

(22) Filed: Jan. 30, 2006

(51) Int. Cl. A61N 5/06

(52) U.S. Cl. 607/94

(57) ABSTRACT

A lens for use on a device to assist in the phototherapeutic treatment of skin disorders where the device includes a housing having an interior containing a source of ultra-violet radiation or being adapted to receive ultra-violet radiation from an external source. The lens comprises a sheet-like structure releasably securable to the housing and for assisting in the direction of ultra-violet radiation from the housing to a desired skin surface. The lens has an upper surface and a lower surface with the upper surface generally adjacent to the interior of the housing. The lens further includes a plurality of generally hollow conical protrusions having upper and lower ends. The protrusions are situated on the lower surface of the lens such that when the lens is secured to the housing ultra-violet radiation is diverted outwardly through the lower ends of the protrusions.

### FIELD OF THE INVENTION

This invention relates to an apparatus or device that may be used to assist in the phototherapeutic treatment of skin diseases or skin disorders which may include psoriasis and other ultra-violet light-responsive dermatoses.

### BACKGROUND OF THE INVENTION

Psoriasis and other similar and related skin disorders affects a statistically significant portion of the population. In the case of psoriasis, an individual experiences an abnormally fast rate of cell proliferation in the basil layer of the epidermis. Left untreated, psoriasis can cause the skin to become red and blotchy, and can eventually lead to the formulation of scaly plates. These plates or patches are often painful and itchy, and can become inflamed and are subject to bleeding and infection. It has been shown that phototherapy provides a method to treat skin psoriasis and other skin disorders and to reduce their symptoms. Phototherapeutic treatment of the skin may involve the application of ultra-violet A (UV-A) and/or ultra-violet B (UV-B) light with or without the application of topical ointments and medications. Due to a number of negative side effects associated with UV-A light treatment, many currently available phototherapeutic methods involve treating effected areas with UV-B light having a wave length generally from about 290 to 320 nm.

A significant difficulty encountered by individuals and medical personnel when treating skin psoriasis and other similar or related disorders with ultra-violet light concerns the difficulty of penetrating hair on the scalp with ultra-violet radiation to treat the skin surface below. Others have proposed the use of brush-like structures that transmit light through solid fibre optic elements or through solid bulbous ends on elongate bristles (see U.S. Pat. No. 2,397,757, dated Apr. 2, 1946, issued to Schwedersky; and U.S. Pat. No. 5,300,097, dated Apr. 5, 1994, issued to Lemer et al). While such devices are generally of assistance in transmitting UV radiation from a source to the scalp of an individual, they also suffer from significant limitations. One such limitation stems from the fact that where fibre optics are utilized, a collimated light source is required where the rays of light travel in essentially parallel paths or columns so that they may be guided into individual fibers without appreciable loss. Often a laser is used as the source of collimated light, adding to the expense of the overall device. In other instances the collimated light source is achieved through the use reflectors and filters to direct the light along the appropriate pathways. To minimize losses, such reflectors and filters must usually be of a relatively high quality, thereby increasing both the complexity and the cost of the equipment within which they are used. Such existing devices are also limited in their ability to clear hair from the skin surface to be treated, for example as is usually necessary when treating the scalp. Current devices rely upon the use of conventional comb attachments that do a relatively poor job of clearing hair out of the way. Such comb devices are also somewhat difficult to use and often permit treatment of only a narrow band of the skin surface, and thereby have a tendency to increase treatment time.

### SUMMARY OF THE INVENTION

The invention therefore provides an apparatus that assists in the phototherapeutic treatment of skin disorders and that is particularly affective in treating skin disorders of the scalp. In one of its applications the device assists in the delivery of ultra-violet light from a scattered or Lambertian light source, such as a fluorescent lamp or tube, to the scalp for the phototherapeutic treatment of psoriasis and other skin disorders.

Accordingly, in one of its aspects the invention provides a lens for use on a device to assist in the phototherapeutic treatment of skin-disorders, the device including a housing having an interior containing a source of ultra-violet radiation or being adapted to receive ultra-violet radiation from an external source, the lens comprising a sheet-like structure releasably securable to the housing and for assisting in the direction of ultra-violet radiation from the housing to a desired skin surface, said lens having an upper surface and a lower surface, said upper surface of said lens generally adjacent to the interior of the housing when said lens is secured thereto, said lens further including a plurality of generally hollow conical protrusions having upper and lower ends, said protrusions situated on said lower surface of said lens such that when said lens is secured to the housing ultra-violet radiation is diverted outwardly through said lower ends of said protrusions.

In a further aspect the invention provides a lens for use on a device to assist in the phototherapeutic treatment of skin disorders, the device including a housing having an interior containing a source of ultra-violet radiation or being adapted to receive ultra-violet radiation from an external source, the lens comprising a sheet-like structure releasably securable to the housing and for assisting in the direction of ultra-violet radiation from the housing to a desired skin surface, said lens having an upper surface and a lower surface, said upper surface of said lens generally adjacent to the interior of the housing when said lens is secured thereto, said lens further including a plurality of generally hollow protrusions having upper and lower ends with openings therethrough, said protrusions situated on said lower surface of said lens such that when said lens is secured to the housing ultra-violet radiation is diverted outwardly through said openings in said lower ends of said protrusions.

In yet a further embodiment the invention concerns a lens for a device used in the phototherapeutic treatment of skin disorders where said treatment includes subjecting a skin surface to irradiation from a source of ultra-violet light, the device of the type including a housing having an interior containing a source of ultra-violet light or being adapted to receive ultra-violet light from an external source, the lens comprised of flexibly resilient material and releasably securable to the housing, said lens having a lower surface and also having an upper surface that is generally adjacent to the interior of the housing when said lens is secured thereto such that ultra-violet light from the source will bombard said upper surface of said lens, said lens including a plurality of conical protrusions on said lower surface, said conical protrusions being generally hollow with hollow interiors and upper and lower ends having openings therethrough so that ultra-violet light cast upon said upper surface of said lens is directed outwardly through said open lower ends of said conical protrusions, said flexible resiliency of said lens allowing said lens to deflect and become generally concave in configuration when a force is applied to said lower surface in a direction toward the housing.

Further aspects and advantages of the invention will become apparent from the following description taken together with the accompanying drawings.

### BRIEF DESCRIPTION OF THE DRAWINGS

For a better understanding of the present invention, and to show more clearly how it may be carried into effect, reference will now be made, by way of example, to the accompanying drawings which show the preferred embodiments of the present invention in which:

FIG. 1 is a schematic view of a phototherapeutic treatment device incorporating an apparatus in accordance with one of the preferred embodiments of the present invention;

FIG. 2 is a side elevational view of the wand or handpiece of the device as shown in FIG. 1;

FIG. 3 is a bottom view of the wand or handpiece shown in FIG. 2;

FIG. 4 is a sectional view taken along the line 4-4 in FIG. 3;

FIG. 5 is a sectional view taken along the line 5-5 in FIG. 3;

FIG. 6 is a sectional view taken along the line 5-5 in FIG. 3 wherein the lens has been displaced inwardly toward the wand or handpiece and is generally concave in shape;

FIG. 7 is an enlarged cross-sectional view of the lens of the wand shown in FIG. 4;

FIG. 8 is a view of an alternate embodiment of the lens of the wand shown in FIG. 1; and,

FIG. 9 is a view of a further alternate embodiment of the lens of the wand shown in FIG. 1.

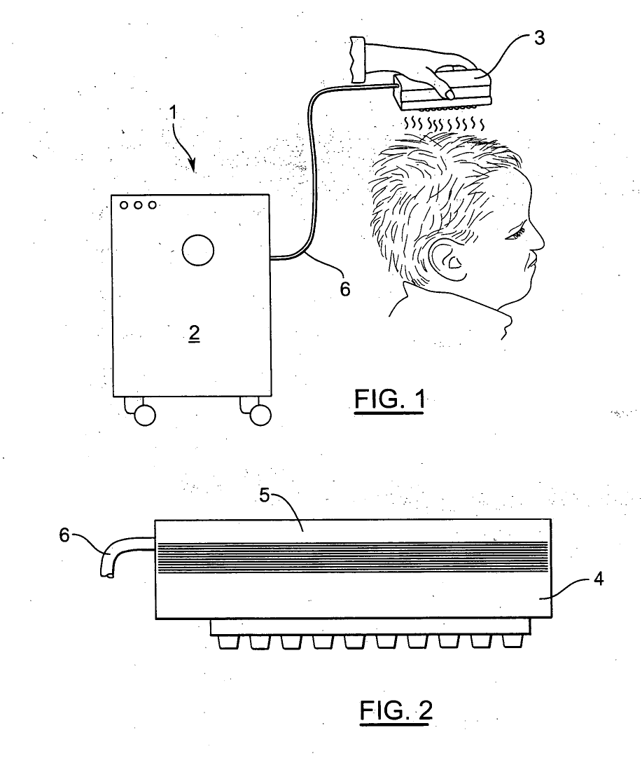

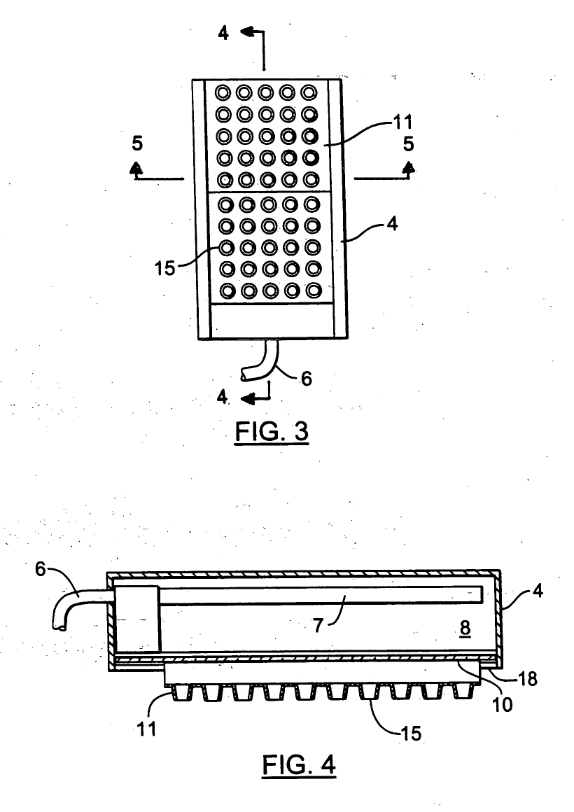

### DESCRIPTION OF THE PREFERRED EMBODIMENT

The present invention may be embodied in a number of different forms. However, the specification and drawings that follow describe and disclose only some of the specific forms of the invention and are not intended to limit the scope of the invention as defined in the claims that follow herein.

FIG. 1 is a schematic view of an example of one form of phototherapeutic treatment device 1 that may incorporate an apparatus constructed in accordance with one of the preferred embodiments of the present invention. In FIG. 1 phototherapeutic device 1 is shown in the form of a portable device that generally comprises a main power unit 2 and a wand or handpiece 3. Depending upon the complexity of the device; and in general whether it is constructed as a consumer based model or in the form of a more robust commercial grade product for use in a medical office or hospital, power unit 2 may include a variety of controls, timers, and other mechanisms that are capable of altering, adjusting or controlling the level and duration of ultra-violet radiation produced.

In most instances wand 3 will be comprised of a housing 4 having an integrated or attached handle 5 allowing the wand to be grasped and easily moved about with the use of one hand. Typically housing 4 will include a generally hollow interior that either contains a source of ultra-violet light or-that is adapted to receive ultra-violet light from an external source. Where the housing contains the source of ultra-violet light in most instances one or more fluorescent tubes or light sources will be situated within the housing's interior. Once again, depending upon the intended end use of device 1, the housing may be connected by means of electrical wires 6 to main power unit 2 or, alternatively, may be plugged directly into an electrical receptacle without the use of the main power unit. In the embodiment shown in the attached figures the source of ultra-violet light is comprised of a number of fluorescent tubes 7 mounted within the hollow interior 8 of housing 4. In an alternate embodiment, the source of ultra-violet radiation may be contained within main power unit 2 with the radiation transmitted to wand 3 through the use of fibreoptic cables or other means of transmission.

In the embodiment of the invention shown in the attached figures wand 3 has a generally opened lower surface or portion that leads into hollow interior 8. Typically a reflector 9 will generally surround fluorescent tubes 7 in order to concentrate and direct radiation generated by the tube outwardly through the open lower portion of the housing. A clear and transparent shield 10 may be installed within the open lower portion of housing 4 to protect fluorescent tubes 7 and reflector 9. Shield 10 would typically be comprised of a sheet of glass, acrylic similar material, however, regardless of its particular construction shield 10 should be of a composition and form that readily permits the transmission of ultra-violet radiation therethrough with relatively minimal losses.

In accordance with the invention there is further provided a lens 11 that is releasably securable to housing 4 and that assists in the direction of ultra-violet light from the housing to a desired skin surface. As shown in the attached drawings, lens 11 is generally in the form of a sheet-like structure 20 having an upper surface 12 and a lower surface 13, with the upper surface generally adjacent the interior of housing 4 and transparent shield 10, where such a shield is utilized. Sheet-like structure 20 may be rectangular, square or any of a wide variety of other shapes depending upon the configuration of housing 4. Typically the lens will be constructed from a relatively thin, flexibly resilient, material. For example, in the attached drawings lens 11 is formed form polyvinyl chloride having a thickness of approximately 0.025 inches. Lens 11 may be releasably secured to housing 4 through the use of a wide variety of different mechanical fasteners which could include screws, bolts, hook and loop fasteners and other such structures. In the embodiment shown, lens 11 includes flanges or flattened portion 14 extending along each of its longitudinal edges for receipt within correspondingly shaped open ended slots 18 within housing 4. This structure permits the lens to be quickly and easily inserted or removed from the slots in the housing, as may be required. For example, in the event that the lens becomes dirty or damaged, for sanitary reasons where wand 3 is to be used for the treatment of different individuals, or where areas of the body are to be treated that do not require the use of the lens, lens 11 may be easily removed from the wand and later replaced when desired. To facilitate the formation and manufacture of 18 and housing 4 it is expected that in most cases the housing will be constructed from extruded metal or plastic.

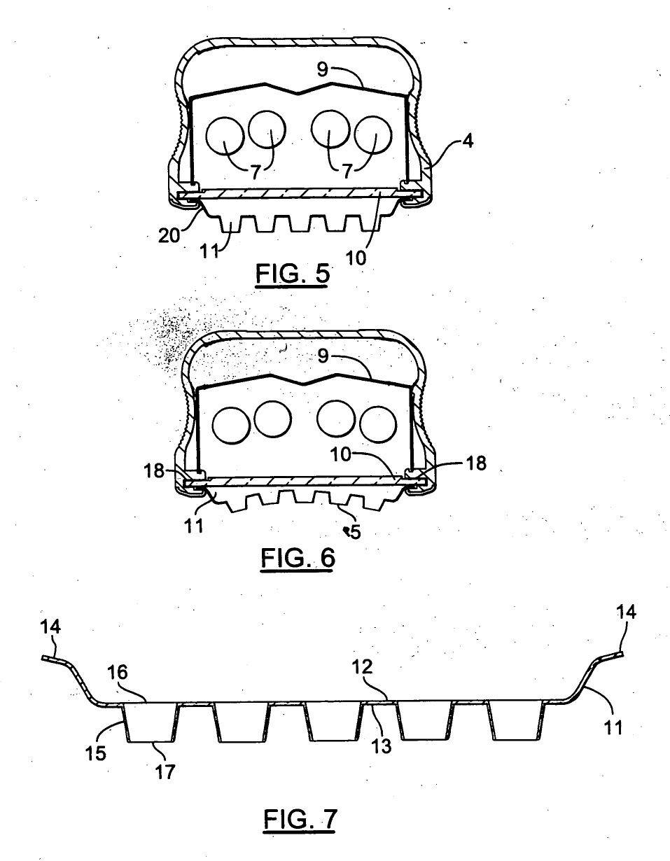

As is shown in the attached drawings, and in particular in FIGS. 5 and 6, lens 11 is preferably off-set slightly from transparent shield 10 so that an airspace or void exists between the lens and the shield. Lens 11 is also preferably formed from a flexibly resilient material such that the application of an external force upon lower surface 13 in a direction toward shield 10 causes the lens to be deflected inwardly toward the shield and to thereby assume a slightly concave configuration. This feature of lens 11 permits it to more closely conform to the curved surface of various parts of the human body. For example, where device 1 is to be used to treat the scalp, the flexibility of lens 11 permits the lens to bend and assume a concave shape and to thereby roughly conform to the radius of the human scalp. The placement of shield 10 in relative close proximity to lens 11 prevents the lens from being displaced into housing 4 beyond a predetermined distance. It has been found that a concavity of lens 11 having a radius of from about 3 to 4 inches presents an effective lens configuration for treatment of the scalp of most individuals. When the force applied to lower surface 13 of lens 11 is removed the resiliency of the lens allows it to return to its original generally flat or slightly convex configuration.

Lens 11 further includes on its lower surface 13 a plurality of generally hollow protrusions 15 having upper ends 16 and lower ends 17 with openings extending therethrough. Protrusions 15 may be of a variety of different shapes and in one preferred embodiment they are generally conical. The conical protrusions provide a funnel-like or conical light pathway such that when upper surface 12 of lens 11 is bombarded with ultra-violet, the radiation tends to be directed outwardly through the open lower ends 17 of protrusions 15. As shown in the attached drawings, conical protrusions 15 are preferably arranged in a series of generally parallel rows along the length of the lower surface of the lens.

It is expected that in many instances lens 11 will be manufactured from a relatively flat sheet of thin plastic material (such as PVC) that is vacuum or pressure thermoformed using a die that creates rows of conical protrusions 15 upon the lens's lower surface. If lens 11 is thermoformed the conical protrusions will in most cases be subsequently precision die cut to form the holes through their lower ends create an array of generally uniform open ended funnel or cone shaped protrusions 15. The conical shape of the protrusions provides them with a relatively significant degree of rigidity, even though the material from which they are constructed is reasonably flexible. At the same time, the arrangement of the protrusions in generally parallel rows presents areas of inherent flexibility between adjacent rows which permits the lens to be deflected inwardly towards shield 10 and assume a slightly concave configuration when an inwardly directed force is applied to the lens's lower surface. As indicated above, this inherent “flexibility” of lens 11 permits it to conform to curved surfaces of the human body, which in turn assists in the delivery of light to the skin surface by helping to position the lower ends of protrusions 15 in close proximity to the treatment area in question. As will be appreciated from a complete understanding of the invention, the rigidity of conical protrusions 15 is such that as the lens is moved across a skin surface containing hair, the protrusions will have a tendency to displace hair from the tips of the cones into areas between adjacent conical protrusions, thereby helping to expose the underlying skin surface and permit a more effective phototherapeutic treatment thereof. Although the conical protrusions may not clear all hair from in front of their open lower ends, it has been found that they clear a sufficient amount of hair to enable better treatment of the skin below than would in most cases be achievable using conventional treatment devices.

In a preferred embodiment of the invention the upper ends 16 of conical protrusions. 15 coincide with the upper surface 12 of lens 11. The openings through the upper and lower ends of the conical protrusions may be circular or rectangular, with the upper surface of the lens and the interior of the protrusions metallized or otherwise rendered reflective to increase their ultra-violet specular reflectivity and minimize UV radiation losses. Metalizing or rendering the upper surface of the lens and the interior of the protrusions reflective also provides ultra-violet protection to the lens substrate material. In one embodiment of the invention lens 11 is preferably opaque such that UV radiation striking upper surface 12 is permitted to exit only through the openings in lower ends 17 of conical protrusions 15. Experimental testing has suggested that where openings through lower ends 17 of conical protrusions 15 are utilized, openings having a diameter of from approximately 2 mm to approximately 10 mm are preferable. It has also been found that conical protrusions of a height of approximately 5 mm and having smooth lower ends enable effective skin treatment with minimized skin irritation. The applicant has also determined that conical protrusions having a height of approximately 5 mm can be formed on lens 11 without significant difficulty through the use of conventional thermoforming processes. It will be appreciated that increasing the height of the conical protrusions will generally have the benefit of permitting a greater volume of hair to be stored between adjacent protrusions. However, a significant increase in the height or length of the conical protrusions may also introduce manufacturing difficulties and may require that other molding and manufacturing processes be utilized.

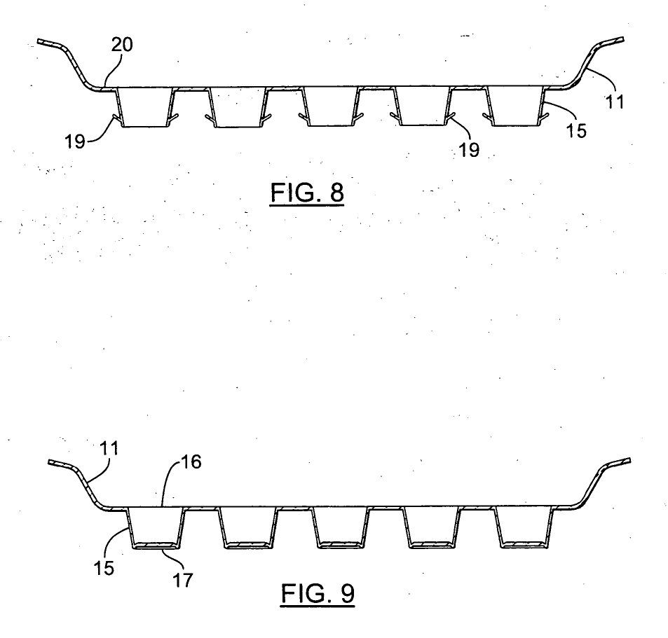

FIGS. 8 and 9 show yet further embodiments of the invention. FIG. 8 shows the inclusion of an annular, outwardly extending, barb 19 situated generally along the lower portion of the outside surface of protrusions 15. Barb 19 improves the ability of lens 11 to collect or grasp hair for receipt into the areas between adjacent protrusions. While a single barb is shown in FIG. 8, multiple barbs could equally be used.

FIG. 9 depicts an embodiment of the invention where conical protrusions 15 have enclosed lower ends without holes therethrough. In this embodiment lens 11 is preferably formed from an ultra-violet transmitting material allowing the lower ends 17 of conical protrusions 15 to remain enclosed. While such a structure may be somewhat less efficient than the embodiment where lower ends 17 have holes through them, conical protrusions without holes should be in most instances less expensive to manufacture.

In operation, wand or handpiece 3 could be used by itself for spot treatment of various areas of the body, such as elbows and knees, or may be used in conjunction with lens 11. It should also be noted that other types of attachments could be inserted into slots 18 in place of lens 11 as may be necessary for specific forms of treatment. Lens 11 will be particularly useful when treating the scalp or other areas of the body covered in hair. In such cases the lens may be inserted into slots 18 of housing 4 without the need to resort to tools or other devices. Where the scalp is to be treated, hair in the affected area is preferably parted first using fingers or a conventional comb or brush after which phototherapeutic device 1 is activated to generate ultra-violet light. The wand with lens 11 in place may then be centered on the parted area of the scalp and moved in a continuous slow circular motion while also slowly moving the lower surface of the lens closer to the scalp. Such movement will tend to generate a force applied to lower surface 13 of lens 11, effectively causing the lens to be displaced towards shield 10 and assume a concave configuration that will generally conform to the scalp. The concave shape of lens 11 helps to maintain an increased number of the conical protrusions in close proximity and at right angles to the scalp. Such a described motion also has the result of tending to displace hair from the tips or lower portions of the conical protrusions into areas between adjacent protrusions and to thereby increase the efficiency of irradiation of the skin surface below. Since the lens is preferably formed from an opaque material, ultra-violet radiation will be permitted to exit only through lower ends 17 of conical protrusions 15 enabling a more accurate control of the application of radiation to the skin. Lens 11 will also help to avoid the needless irradiation of hair when treating the scalp as significant portions of the hair over the treatment area will be stored between the protrusions 15.

It will thus be appreciated that the structure of lens 11 as described above allows for the efficient irradiation of a skin surface through the utilization of a low cost fluorescent light source and that there is no necessity for the use of collimated light. Further, it will also be appreciated that the reflective hollow cone structure of conical protrusions 15 permits ultra-violet radiation to exit through the lower ends of the protrusions with minimal loss since the radiation travels through air only after being transmitted through shield 10 if a shield is utilized. Utilizing hollow conical protrusions on lens 11 thereby presents a means to concentrate ultra-violet radiation with minimal transmission losses and at minimal cost.

It is to be understood that what has been described are the preferred embodiments of the invention and that it may be possible to make variations to these embodiments while staying within the broad scope of the invention. Some of these variations have been discussed while others will be readily apparent to those skilled in the art.

I claim:

1. A lens for use on a device to assist in the phototherapeutic treatment of skin disorders, the device including a housing having an interior containing a source of ultra-violet radiation or being adapted to receive ultra-violet radiation from an external source, the lens comprising:
a sheet-like structure releasably securable to the housing and for assisting in the direction of ultra-violet radiation from the housing to a desired skin surface, said lens having an upper surface and a lower surface, said upper surface of said lens generally adjacent to the interior of the housing when said lens is secured thereto, said lens further including a plurality of generally hollow conical protrusions having upper and lower ends, said protrusions situated on said lower surface of said lens such that when said lens is secured to the housing ultra-violet radiation is diverted outwardly through said lower ends of said protrusions.

2. The lens as claimed in claim 1 wherein said lower ends of said protrusions have holes or openings therethrough.

3. The lens as claimed in claim 2 wherein said conical protrusions are arranged in a series of generally parallel rows along said lower surface of said lens.

4. The lens as claimed in claim 2 being flexibly resilient such that an application of force against said lower surface causes said lens to deflect and become generally concave.

5. The lens as claimed in claim 2 wherein said conical protrusions are rigid such that when said lens is moved across a skin surface containing hair at least a portion of the hair is displaced into voids between adjacent conical protrusions.

6. The lens as claimed in claim 2 wherein said upper surface of said lens and the interior of said generally hollow conical protrusions are reflective.

7. The lens as claimed in claim 2 wherein said upper ends of said conical protrusions generally coincide with said upper surface of said lens.

8. The lens as claimed in claim 2 wherein said openings through said lower ends of said conical protrusions are from approximately 2 mm to approximately 10 mm in diameter.

9. The lens as claimed in claim 1 wherein said conical protrusions include one or more outwardly extending barbs on their exterior surfaces.

10. The lens as claimed in claim 2 formed from a generally opaque material.

11. A lens for use on a device to assist in the phototherapeutic treatment of skin disorders, the device including a housing having an interior containing a source of ultra-violet radiation or being adapted to receive ultra-violet radiation from an external source, the lens comprising:
a sheet-like structure releasably securable to the housing and for assisting in the direction of ultra-violet radiation from the housing to a desired skin surface, said lens having an upper surface and a lower surface, said upper surface of said lens generally adjacent to the interior of the housing when said lens is secured thereto, said lens further including a plurality of generally hollow protrusions having upper and lower ends with openings therethrough, said protrusions situated on said lower surface of said lens such that when said lens is secured to the housing ultra-violet radiation is diverted outwardly through said openings in said lower ends of said protrusions.

12. The lens as claimed in claim 11 wherein said protrusions are generally conical in shape.

13. The lens as claimed in claim 11 being flexibly resilient such that an application of force against said lower surface causes said lens to deflect and become generally concave.

14. The lens as claimed in claim 11 formed from a generally opaque material.

15. The lens as claimed in claim 11 wherein said upper surface of said lens and the interior surface of said hollow protrusions are reflective.

16. The lens as claimed in claim 11 wherein said protrusions include one or more outwardly extending barbs on their exterior surfaces.

17. The lens as claimed in claim 11 wherein said protrusions are rigid such that when said lens is moved across a skin surface containing hair at least a portion of the hair is displaced into voids between adjacent protrusions.

18. The lens as claimed in claim 11 wherein said openings in said lower ends of said protrusions are from approximately 2 mm to approximately 10 mm in diameter.

19. A lens for a device used in the phototherapeutic treatment of skin disorders where said treatment includes subjecting a skin surface to irradiation from a source of ultra-violet light, the device of the type including a housing having an interior containing a source of ultra-violet light or being adapted to receive ultra-violet light from an external source, the lens comprised of flexibly resilient material and releasably securable to the housing, said lens having a lower surface and also having an upper surface that is generally adjacent to the interior of the housing when said lens is secured thereto such that ultra-violet light from the source will bombard said upper surface of said lens, said lens including a plurality of conical protrusions on said lower surface, said conical protrusions being generally hollow with hollow interiors and upper and lower ends having openings therethrough so that ultra-violet light cast upon said upper surface of said lens is directed outwardly through said open lower ends of-said conical protrusions, said flexible resiliency of said lens allowing said lens to deflect and become generally concave in configuration when a force is applied to said lower surface in a direction toward the housing.

20. The lens as claimed in claim 19 formed from a generally opaque material.
